(19) United States
(12) Patent Application Publication
(10) Pub. No.: US 2010/0168524 A1
(43) Pub. Date: Jul. 1, 2010

(54) SYSTEM AND METHOD FOR HEART FAILURE PREDICTION

(75) Inventors: Gadi Cotter, Chapel Hill, NC (US); Beth Davison Weatherley, Chapel Hill, NC (US)

Correspondence Address: Leason Ellis LLP, 81 Main Street, Suite 503, White Plains, NY, 10601, US

(73) Assignee: Momentum Research Inc., Durham, NC (US)

(21) Appl. No.: 12/346,439

(22) Filed: Dec. 30, 2008

(51) Int. Cl. A61B 5/00

(52) U.S. Cl. 600/300

(57) ABSTRACT

A method for monitoring a health status of a human subject includes the capturing of medical data concerning the health of the subject at defined intervals using a questionnaire. The questionnaire provides a standard script for data capture. Part of the captured data is constrained to a Likert scale while other data is on a visual analog scale. The captured data further includes an assessment by a physician of health symptoms of the subject. The captured data is input into a computer, provided to an algorithm that is configured to assess a risk of acute heart failure. The risk of acute heart failure is computed using the algorithm and the captured data from a plurality of the defined intervals. In one method, the health status of the subject as being either improved or worsening is output to the physician as a function of a value of the computed risk. In another method, a survival function outcome for the subject is predicted using the output of the algorithm.

### FIELD OF THE INVENTION

The present invention relates to the field of medicine and more particularly concerns systems and methods configured to predict a risk of heart failure in a patient or drug candidate.

### BACKGROUND OF THE INVENTION

Heart failure is a condition in which the blood flow throughout the body is not adequate to maintain the metabolic requirements. Heart failure does not mean that the heart has stopped or is about to stop working. This deficiency in the cardiovascular system can cause, among other things, blood and fluid to back up into the lungs, a buildup of fluid in the feet, ankles and legs (an ailment called edema), and tiredness and shortness of breath (an ailment known as dyspnea). The leading causes of heart failure are coronary artery disease, high blood pressure and diabetes. Treatment includes treating the underlying cause of your heart failure, medicines, and heart transplantation if other treatments fail. Heart failure is a serious condition. About 5 million people in the U.S. have heart failure. It contributes to 300,000 deaths each year.

There are several health symptoms or ailments that are potentially attributable to heart failure, though they could be manifestations of other medical issues or normalcy in certain individuals. Medical practitioners use their understanding, skill and experience to diagnose heart failure in view of clinic data for the individual, but there is no uniformity in diagnoses. Among the symptoms and ailments considered by such practitioners are the following.

Dyspnea, a shortness of breath, occurs normally during intense physical exertion or at high altitude, but can occur at other times in an individual whose heart is not healthy. Generally, dyspnea is the individual's subjective apprehension of difficulty or distress in breathing. In the absence of physical exertion or high altitudes, this symptom is most commonly associated with disease of the heart or lungs.

Orthopnea is a form of dyspnea that occurs when lying flat. Persons suffering from orthopnea must sleep propped-up in bed or sitting in a chair so as to not be awakened. It is commonly measured according to the number of pillows needed to prop the patient up to enable breathing (Example: “3 pillow orthopnea”).

Edema, formerly known as dropsy or hydropsy, is an abnormal accumulation of fluid beneath the skin, or in one or more cavities of the body. Edema is often more prominent in the lower legs and feet toward the end of the day as a result of pooling of fluid from the upright position usually maintained during the day. Upon awakening from sleeping, people can have swelling around the eyes referred to as periorbital edema. More generally, edema can be caused by an increased secretion of fluid into the interstitium or impaired removal of this fluid relative to the balance of fluid homeostasis.

Rales are the clicking, rattling, or crackling noises heard on auscultation of (listening to) the lung with a stethoscope during inhalation. The sounds are caused by the “popping open” of small airways and alveoli collapsed by fluid, exudate, or lack of aeration during expiration. The word “rales” derives from the French word râle meaning “rattle.” Among other causes, rates can be heard in patients with pulmonary edema secondary to left-sided congestive heart failure.

The jugular venous pulse (JVP, sometimes referred to as jugular venous pressure) is the indirectly observed pressure over the venous system. It can be useful in the differentiation of different forms of heart and lung disease. An elevated JVP is the classic sign of venous hypertension (e.g. right-sided heart failure). JVP elevation can be visualized as jugular venous distension, whereby the JVP is visualized at a level of the neck that is higher than normal. To visualize JVP, the patient is positioned under 45°, and the filling level of the jugular vein determined. Although there is some controversy, either the internal or external jugular vein may be used, with the external preferred. In healthy people, the filling level of the jugular vein should be a maximum of several (3-4) centimeters above the sternal angle. A pen-light can aid in discerning the jugular filling level by providing tangential light. The JVP is easiest to observe if one looks along the surface of the sternocleidomastoid muscle, as it is easier to appreciate the movement relative the neck when looking from the side (as opposed to looking at the surface at a 90 degree angle). Like judging the movement of an automobile from a distance, it is easier to see the movement of an automobile when it is crossing one's path at 90 degrees (i.e. moving left to right or right to left), as opposed to coming toward one.

Clinicians use any or all of the foregoing in their interpretation of the individual's risk of heart failure in connection with treatment and sometimes in determining whether a person is a candidate for a particular drug therapy. However, being able to provide a standardized method for predicting heart failure in patients or drug candidates would be a desirable advance in the art, and so would a system for doing same. The present invention addresses this deficiency in the art.

### SUMMARY OF THE INVENTION

According to one aspect of the invention, a method for monitoring a health status of a human subject includes the capturing of medical data concerning the health of the subject at defined intervals using a questionnaire. The questionnaire provides a standard script for data capture. Part of the captured data is constrained to a Likert scale while other data is on a visual analog scale. The captured data further includes an assessment by a physician of health symptoms of the subject and some laboratory evaluations. The captured data is input into a computer, provided to an algorithm that is configured to assess a risk of patients with acute heart failure. The risk of the patients with acute heart failure is computed using the algorithm and the captured data from a plurality of the defined intervals. The health status of the subject as being either improved or worsening is output to the physician as a function of a value of the computed risk.

According to another aspect of the invention, a method for predicting a survival function outcome of a human subject includes the capturing of medical data concerning the health of the subject at defined intervals using a questionnaire. The questionnaire provides a standard script for data capture. Part of the captured data is constrained to a Likert scale while other data is on a visual analog scale. The captured data further includes an assessment by a physician of health symptoms of the subject. The captured data is input into a computer, provided to an algorithm that is configured to assess a risk of acute heart failure. The risk of acute heart failure is computed using the algorithm and the captured data from a plurality of the defined intervals. A survival function outcome for the subject is then predicted using the output of the algorithm.

These and other aspects, features, and advantages of the present invention will be appreciated from the accompanying description of certain embodiments of the invention and the drawing figures included herewith.

### BRIEF DESCRIPTION OF THE DRAWING FIGURES

FIGS. 1A and 1B comprise a flow diagram showing patient assessment steps as well as certain processes performed by a system and method in accordance with an embodiment of the invention;

FIG. 2A is an example of a part of a questionnaire that can be used in accordance with an embodiment of the invention to coordinate patient data with a specific observation;

FIG. 2B is an example of another part of the questionnaire of the embodiment of FIG. 2A suitable for capturing subjective data on a Likert scale, more particularly, for capturing dyspnea data relative to the beginning of a patient study;

FIG. 2C continues the example questionnaire of FIGS. 2A and 2B showing a part of the questionnaire that is suitable for capturing subjective data on a visual analog scale, more particularly, for capturing dyspnea data concerning how a person perceives their breathing at the time of the present observation;

FIG. 2D shows another part of the questionnaire that is arranged to capture general well being data relative to the beginning of the patient study on a Likert scale;

FIG. 2E shows another part of the questionnaire that is arranged to capture general well being data relative to the individual's life history on a visual analog scale;

FIG. 2F shows another part of the questionnaire that is arranged to capture a clinician's observations and lab data concerning the individual; and

FIG. 3 is a block diagram of a system in accordance with an embodiment of the invention.

### DETAILED DESCRIPTION OF CERTAIN EMBODIMENTS OF THE INVENTION

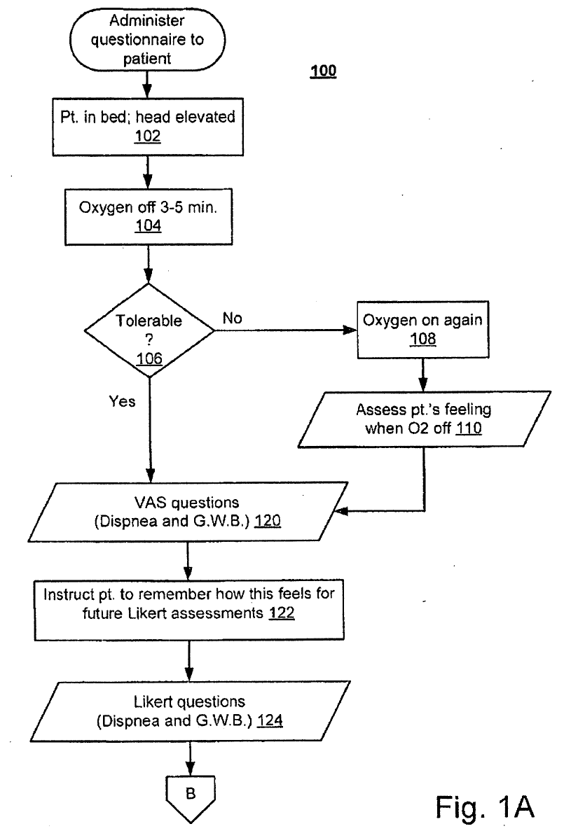

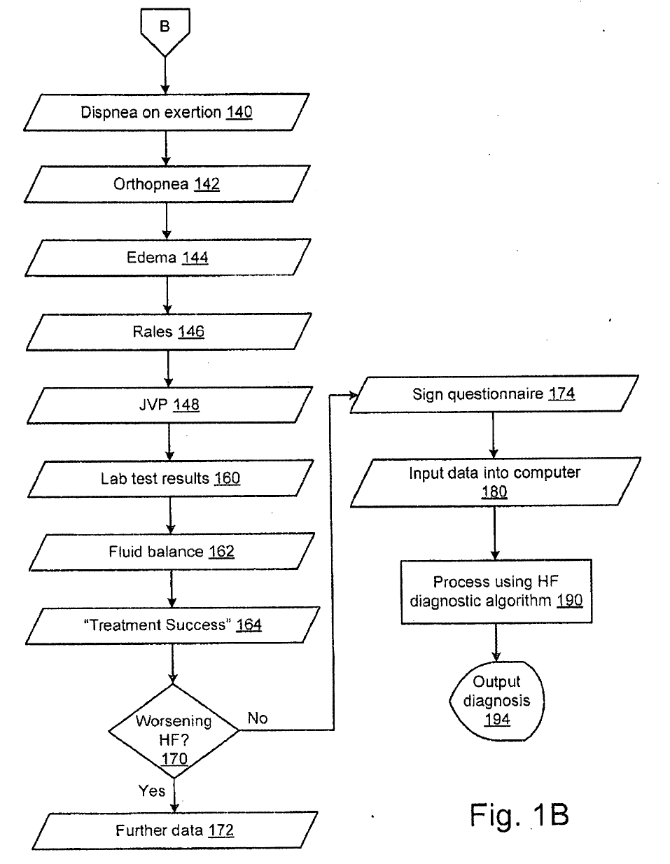

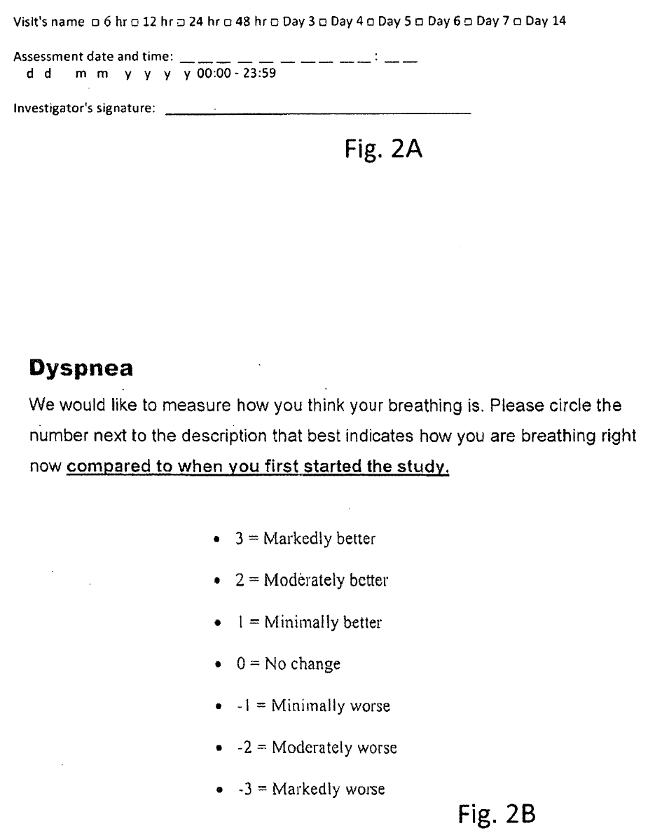

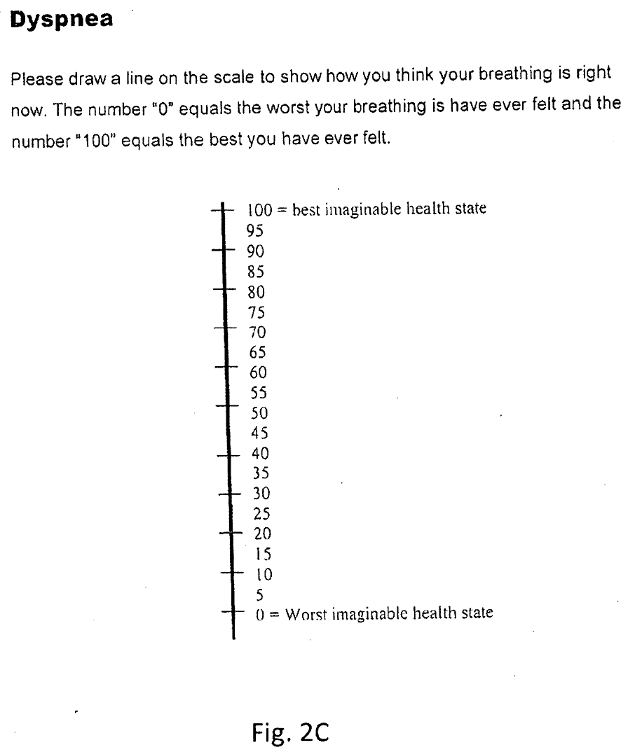

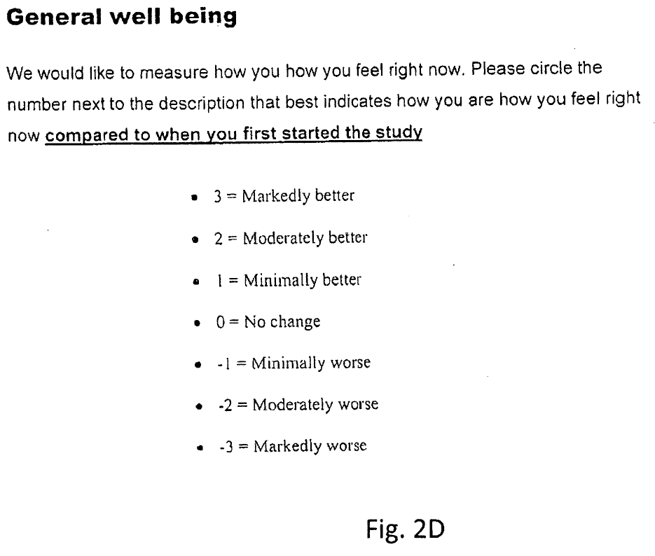

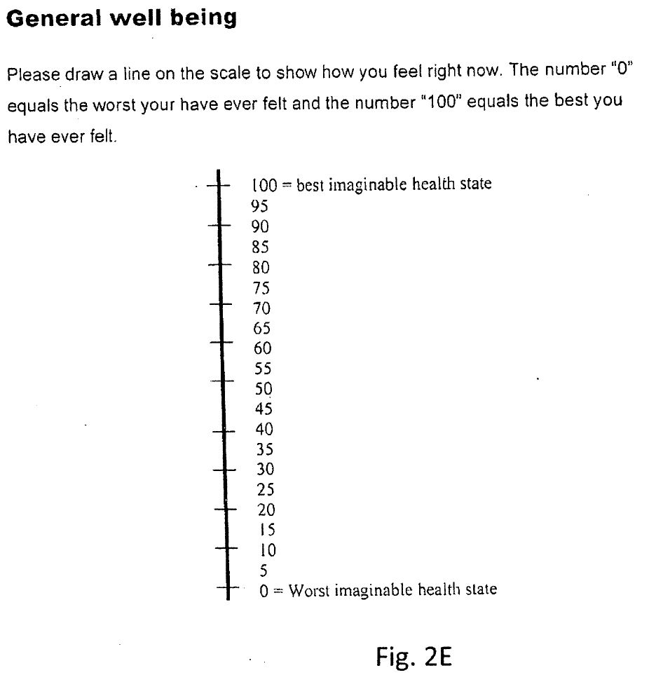

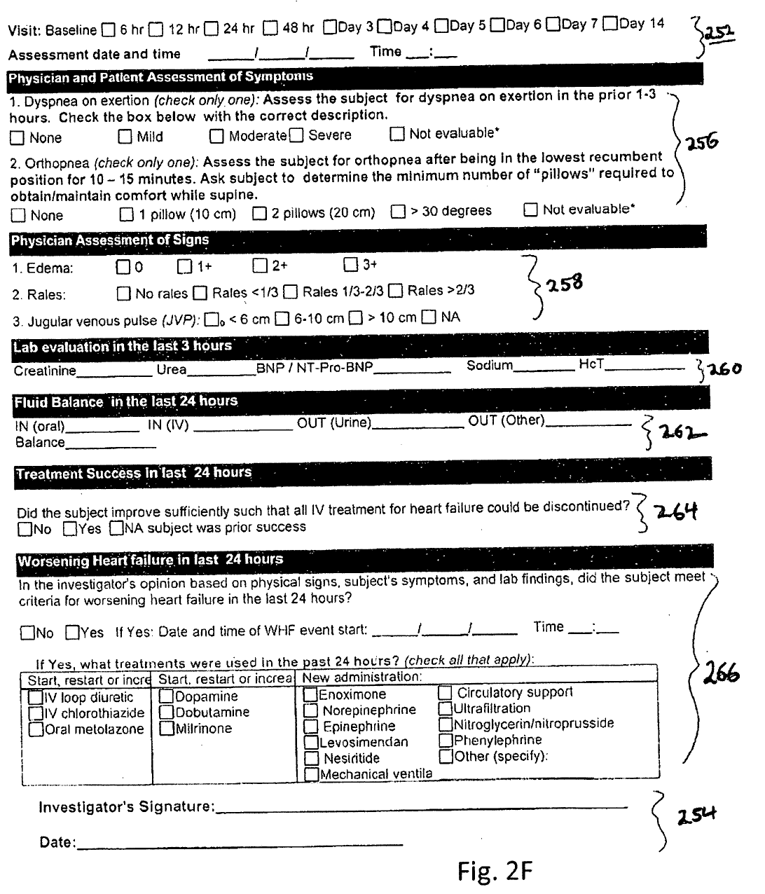

Referring first to FIG. 1A, a flow diagram illustrates a series of steps taken by medical staff in connection with gathering subjective data on a patient's condition. The staff member has a questionnaire that is presented to the patient once desired conditions are achieved, and that data is later used in an algorithm, discussed below, to diagnose a risk of heart failure for that patient. The information is preferably gathered using a questionnaire as shown in FIGS. 2A through 2F, but the invention is not so limited.

At block 102, the staff member prepares to capture an assessment of the patient's dyspnea and general well being (GWB) and checks that the patient is lying in bed with his or her head elevated at approximately a 30°, such as by pillows or by articulating the bed itself. The staff member also turns off any oxygen supplement that may have been supplied to the patient and waits from about 3 to about 5 minutes before starting the assessment, as indicated at block 104. Some patients will not tolerate the lack of an oxygen gas supplement (which is purpose of the conceptual test at block 106). For instance, the patient could indicate that he or she cannot breathe effectively. The staff member is trained to monitor for this and if the patient cannot tolerate the lack of oxygen, then the oxygen supply is restored to the patient, as indicated at block 108, and the staff member is to immediately ask the patient to describe how he or she felt when the oxygen was off, as indicated at block 110. This information is captured on the questionnaire.

The questionnaire preferably has a first part that is completed with answers provided by the patient concerning his or her subjective feelings, and a second part completed by a physician or other medical staff. FIGS. 2A through 2E concern the first portion of the questionnaire. Preferably, a new worksheet is completed for each assessment time point through at least day 7 or the day of patient discharge.

The questionnaire is preferably administered even after the subject patient is no longer receiving a study drug.

Referring now to FIGS. 2A through 2E, a preferred arrangement for a questionnaire comprises a worksheet that can be administered in a standardized manner to obtain the patient's subjective assessment of his or her dyspnea and general well being. It should be understood that portions or all of the worksheets can be made available as interactive electronic pages that are arranged in similar manner to capture data using a standard script as described in connection with FIG. 2, and that a paper questionnaire is for purposes of illustration only. Thus, the questionnaire can take on a variety of forms so long as it is configured to capture data at defined intervals and so long as it provides a standard script for sequential data capture. In the preferred implementation described herein, a first portion of the captured data is captured on a Likert scale whereas a second portion of the captured data is on a visual analog scale.

Referring briefly to FIG. 2A, the staff member administering the questionnaire identifies the visit to the patient using a form that prompts for identification of the date and time of the visit and which visit it is. The patient is assessed at intervals from an initial visit (referred to herein as the baseline assessment or “baseline”) and extending over a seven day or more periods. The intervals can be spaced by unequal amounts of time. Thus, for instance, the interval from the first assessment to the next one can be about 6 hours. The intervals can then be six hours later still (at the 12 hour mark), about 12 hours the second interval, and then about 24 hours between each successive interval over a week or so. The staff member optionally signs the form to identify himself or herself and affirm the contents of the entries.

For the initial visit, e.g., before a first dose of a study drug is started on the patient, the staff member only asks the patient the questions from the questionnaire that call for a visual analog scale (VAS). VAS-style dyspnea questions are more sensitive to short term changes (e.g., hours) in a patient's health and more probative of long term changes (e.g., days). No questions in the Likert format are asked because the first visit establishes the “baseline” for such questions. Thus, at block 120, during an initial visit, the staff member will ask the patient to answer the VAS questions, preferably as shown in FIGS. 2C and 2E. In particular, the staff member is preferably instructed to ask the questions (or have the patient read along) exactly as provided in the questionnaire, or any official certified translation thereof. Thus, in the example of FIGS. 2C and 2E, the staff member asks the patient “How is your breathing right now as compared to how you have ever felt?” (a dyspnea question) and “How you are/feel right now as compared to how you have ever felt?” (a GWB question). In response to each of these questions, the patient is encouraged to personally mark their response on a visual analog scale, such as from 0 to 100. The staff member should not coach the patient toward any particular location on the scale, but rather encourage the patient to select a place on the scale that is the most appropriate response from the patient's point of view. As indicated at block 122, the staff member should instruct the patient to remember how this feels because at the next visit (e.g., 6 hours after the initial visit), the Likert assessments will be added to the patient assessment.

A Likert scale (pronounced ‘lick-urt’) is a type of psychometric response scale often used in questionnaires, and is the most widely used scale in survey research. When responding to a Likert questionnaire item, respondents specify their level of agreement to a statement. Likert scaling is a bipolar scaling method, measuring either positive or negative response to a statement. Likert scales may be subject to distortion from several causes. Respondents may avoid using extreme response categories (central tendency bias); agree with statements as presented (acquiescence bias); or try to portray themselves or their organization in a more favorable light (social desirability bias). After the questionnaire is completed, each item may be analyzed separately or in some cases item responses may be summed to create a score for a group of items. Hence, Likert scales are often called summative scales. Likert-style dyspnea questions are more sensitive to short term changes in a patient's health and less probative of long term changes.

In the examples shown in FIGS. 2B and 2D, the Likert questions have a scale of ±3. As indicated at block 124, during each assessment (after the initial assessment), the staff member asks further questions concerning the patient's breathing ability and general well being. Thus, in the example of FIG. 2B, the staff member asks the patient “How is your breathing as compared to when you started the study?” (a dyspnea question), and the patient is to recall how he or she felt at the beginning of the study and provide a comparative answer that the staff member records in the questionnaire. The patient can either mark the answer on the questionnaire himself or herself, or the answer can be conveyed verbally to the investigator who then marks it for the patient. In the example of FIG. 2C, the staff member asks the patient “How you are/feel right now as compared to when you started the study?” (a GWB question), and in response the patient is to recall how he or she felt at the beginning of the study and provide a comparative answer that the staff member records in the questionnaire. Again, for Likert-type questions, either the patient or the staff member can mark the patient's answer on the questionnaire. The staff member should not coach the patient toward any particular location on the scale.

The foregoing steps capture data from the patient's perspective with respect to the patient's breathing and general well-being. These assessments are preferably recorded daily until discharge or day 7, which ever is later. A separate form can be used for each assessment on each day, with the visit being identified such as described above in connection with FIG. 2A. All of the information that is captured is input into a computer, either after administering the questionnaire, as in the case of a paper-based questionnaire, or concurrently with data capture, as in the case of a computer-implemented questionnaire.

In accordance with a salient aspect of the invention, predictions can be made of a patient's risk of heart failure, such as in the form of a sixty-day outcome assessment, using a smaller set of data collected after three to seven days of monitoring. In part, the assessment is based on the patient's responses, but also on an algorithmic processing of physician- or lab-supplied data. The captured data including the physician- and/or lab-supplied data is provided to an algorithm executing within a processor of a computer. The algorithm is configured to assess a risk of acute heart failure through its execution, as described further below. Such predictions improve and essentially optimize the ability of a physician to monitor changes in a patient's status and response to treatment and take action as necessary or possible to preserve life.

Referring now to FIG. 2F, a further component of the questionnaire of FIGS. 2A through 2E is illustrated, though this component can be independent of the other components that comprise the questionnaire. The questionnaire of FIG. 2F is for completion by a physician based on a structured assessment of certain health symptoms, signs, lab evaluations, and an overall assessment by the physician of whether the patient exhibits worsening heart failure as compared to the previous 24 hour period. As in FIG. 2A, the physician identifies the visit using by indicating the date and time of the visit and which visit it is, as shown at the top of the form, such as in a section 252. The physician optionally signs and dates the form at the bottom, such as in a section 254, to identify himself or herself and affirm the contents of the entries. The health symptoms of the subject (e.g., a patient), as will be appreciated from the discussion below, are represented by data values that are used by an algorithm to assess a patient's status or to predict a survival function.

The form of FIG. 2F is arranged to capture several different data points that are used in the assessment of the patient's risk of heart failure. One set of data that is input to the form and captured for providing to the algorithm described below is an assessment of the patient's symptoms. In this regard, the patient is assessed in terms of dyspnea on exertion and orthopnea, preferably in the prior one to three hours. As shown in section 256, the physician rates dyspnea on exertion on a scale of none to severe or not-evaluable. The criteria for this rating are defined, and in one embodiment can be the scores as provided by the NYHA, as shown in the table below.

| SCORE | SYMPTOM |
|---|---|
| None | No limitation: Ordinary physical activity does not cause |
| | Dyspnea |
| Mild | Slight limitation of physical activity: Comfortable at rest. |
| (NYHA II) | Ordinary physical activity results in dyspnea |
| Moderate | Marked limitation of physical activity: Comfortable at |
| (NYHA III) | rest, less than ordinary activity leads to symptoms |
| Severe | Inability to carry on any physical activity without |
| (NYHA IV) | discomfort: Dyspnea present even at rest. With any |
| | physical activity, increased discomfort is experienced. |
| Not evaluable | Unable to determine due to subject's condition |
| | (e.g., too ill to respond or immobile for other reason) |

The orthopnea assessment is preferably performed after the patient has been in the lowest recumbent position permissible (e.g., flat) for about ten to about fifteen minutes. The test proceeds by assessing through interrogation and monitoring of the patient the minimum number of “pillows” required to obtain or maintain comfort while supine. The “pillow” test translates as follows: None, 1 pillow (10 cm), 2 pillows (20 cm), >30 degrees, or not evaluable (e.g., the patient cannot be positioned or maintained supine for the requisite period of time before performing the test). The test result is recorded in section 256.

Next, data points are captured to assess signs exhibited by the subject. As shown in section 258, the physician records the results of a brief physical examination of the subject. As illustrated, three tests are performed and the results are recorded on the form of FIG. 2F. The order of the tests can be varied. One of the tests has the physician recording any signs of edema. This data is captured such as by examining any dependent area, including the lower extremities or the sacral region of the subject. The signs are recorded on a scale of 0 to 3, using well-known and defined criteria such as shown in the table below:

| SCORE | SIGNS OBSERVED |
|---|---|
| 0 | Complete absence of skin indentation with mild digital |
| | pressure in all dependent areas |
| 1+ | Indentation of skin that resolves over 10-15 seconds |
| 2+ | Indentation of skin is easily created with limited pressure and |
| | disappears slowly (15-30 seconds or more) |
| 3+ | Large areas of indentation easily produced and slow to |
| | resolve (>30 seconds) |

The physician also records the results of a rates test, after auscultation of the longs with a stethoscope during inhalation. In one embodiment a four point scale can be applied using the criteria indicated in the table below:

| SCORE | SIGNS OBSERVED |
|---|---|
| 0 | No rales after clearing with cough |
| <⅓ | Moist or dry rales heard in lower ⅓ of one or both lung |
| | fields that persist after cough |
| <⅓-⅔ | Moist or dry rales heard throughout the lower ⅓-⅔ |
| | of one or both lung fields |
| >⅔ | Moist or dry rales heard throughout both lung fields |

The physician also records the results of a jugular venous pulse (JVP) test by measuring in centimeters the vertical distance from the top of any pulsation in jugular veins to the sternal angle of Louis. The test is preferably performed with the subject supine at approximately 45 degrees off the horizontal until the jugular venous pulsation is visible half-way up the neck. In one embodiment, a four point scale is applied using the criteria indicated in the table below:

| SCORE | | SIGNS OBSERVED |
|---|---|---|
| <6 | cm | Complete absence of discernable venous wave, even |
| | | with hepatic pressure. |
| 6-10 | cm | Venous wave detectable during expiration or complete |
| | | respiratory cycle, <4 cm above clavicle (<10 cm). |
| >10 | cm | Presence of venous wave throughout respiratory cycle, |
| | | sometimes ≧4 cm above clavicle and increased with |
| | | hepatic compression. |

| | |
|---|---|
| Not evaluable | Unable to determine due to subject's body build |
| | (habitus) or other reason |

The physician should also enter into the form of FIG. 2F lab test results that are associated with the subject, such as on a patient chart. Among the lab evaluations are any results from preceding three-hour period concerning creatinine, urea, BNP/NT-Pro-BNP (b-type natriuretic peptide/N-terminal pro b-type natriuretic peptide), sodium, and hematocrit (HcT) levels. These tests, included in section 260, may or may not have been performed in the preceding three-hour period, but are useful to the physician in differentiating between heart failure and other problems, such as lung disease. Certain fluid levels also can be recorded over the preceding 24-hour period and captured on the form in section 262. Among relevant fluid balance information is the amount of fluids in orally and intravenously, the amount of urine from the subject, the amount of any other fluids coming from the subject, and a calculation of the overall fluid balance.

In section 264, the physician indicates whether there has been treatment success in the last 24-hour period. The criteria are whether the subject improved sufficiently such that all intravenous (TV) treatment for heart failure could be discontinued. Thus, the physician is to answer “Yes” if the subject's heart failure symptoms have improved enough for all IV therapies to be discontinued or changed to an oral form. A “yes” can be recorded even if an TV diuretic is continuing for reasons other than subject status. This section should be marked “N/A” if treatment success had previously been reported. For all other circumstances, the answer to be marked is “no.”

In section 266, the physician indicates whether there has been a worsening of the subject's heart failure condition in last 24-hours. This question seeks the physician's opinion, based on physical signs and symptoms recorded as a result of the questionnaire presented to the patient and as recorded in sections 256 through 264, whether the subject experienced worsening heart failure in the last 24 hours. If the answer to this is yes, then the date and time of the worsening heart failure (WHF) event are recorded, and the treatments applied in the past 24-hours are to be noted. The treatments may include, for instance, an increase in dose or restart of IV diuretic, an implementation of mechanical respiratory of circulatory assist measures (including BiPAP and CPAP), an administration of IV positive inotropes, or vasopressors, and so on.

In the event that one or more values are missing, they can be replaced by other values in the database for the same patient. For instance, in the case of missing dyspnea values (Likert or VAS), the prior value can be carried forward from the last observation (questionnaire) or it can be interpolated by a weighted method using the last available, previous, and/or succeeding values. As another example, missing serum creatinine data can be replaced by carrying forward the last value or interpolation as just described. In this way, the algorithm used to predict a survival function outcome for the human subject can proceed even on the basis of incomplete data (which is also tolerated by the Kaplan-Meier estimator).

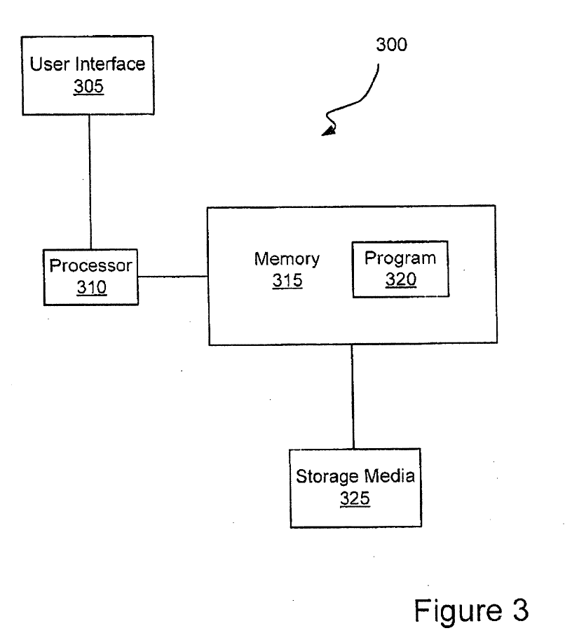

At least a portion of the data captured on the form of FIG. 2F, is provided to an algorithm that comprises part of a program 320 executing within a machine such as a computer or server. A computer system 300 that can implement an embodiment of the invention is described in connection with FIG. 3 below. The algorithm utilized by the program 320 is discussed next. The algorithm utilizes the information garnered using the questionnaire to determine whether a given subject is a “success” or a “failure.” A failure can be characterized by the patient having died, or being assessed as having WHF, or as having renal impairment as evinced by an increase in serum creatinine over the baseline measurement of 0.3 or greater. A success in accordance with the invention requires a measurement of at least 2 on a Likert scale of ±3 (i.e., moderately better), and also must not be a “failure.” Thus, a success has four components: a Likert dyspnea score of at least 2, the patient not being dead, not having worsened heart failure, and not having an increase in serum creatinine over the baseline measurement by 0.3 or greater.

More particularly, the algorithm considers the day-2 and day-3 questionnaire results for the Likert dyspnea test for subjects that do not have a treatment failure by day 7 (i.e., for subjects who are still alive, who do not have WHF), and who do not have an increase in serum creatinine over the baseline measurement by 0.3 or greater by day-5, day-7, or day-14. Thus, the algorithm uses the data captured and provided to it to process the raw data and transform it into a value. The algorithm outputs are double. First, it gives the clinician a more accurate way to assess the progression of the patient's condition. Second it facilitates the evaluation of the effect of different treatments on the patient's status. The tool can also be use for prediction of the likelihood of death or re-hospitalization by day-60 using this information. The prediction follows a Kaplan-Meier survival function and, as such, is an estimator of the survival likelihood of particular subjects having treatment success in the absence of failure in accordance with the algorithm.

In one particular embodiment, the algorithm utilizes three scores in connection with its computations and in generating its output. A first score is based on the day-2 and day-3 Likert dyspnea test (“1^{st} value”). A success, as described above, can provide a value such as “1.” Another score is the lack of a failure, i.e., no death and no WHF by day-7 and serum creatinine remaining within 0.3 of the baseline measurement by day-5, day-7, or day-14 (“2^{nd} value”). The lack of a failure can be accorded a score of “0.” In addition, a score can be recorded on a relative scale of VAS-dyspnea recordings taken day-over-day, that is, day 0 to day 1, day 1 to day 2, or over larger periods such as day 0 to day 2, etc. (“3^{rd} value”). An average change in the absolute number from day-to-day measurements may not be large, since all values are on the same scale. This average can be computed by the processor 310 (discussed below) and used by the algorithm. For instance, 3^{rd} value might be “9.” The values “1,” “0” and “9” can be combined in a weighted sum or area under the curve measurement to put more emphasis on success and still more emphasis a non-zero failure value with the output governing whether the system indicates improvement or worsening of the patient at that stage of the treatment and in view of the captured data available so far.

In the event that the subject experienced a failure in the determination of the physician, any VAS value recorded by the patient can be overridden with an imputed value, such as a value associated with the worst possible condition e.g., a very low number in the range of 0-5. These values are important in determining the changes in a given patient's status and his or her response to treatment. For instance if a patient develops failure (2^{nd} value) and has no success (1^{st} value) and no positive improvement in 3^{rd} value, then a system implementing the algorithm described above can output for the clinician a determination that the patient's status has deteriorated and the patient did not respond to a given therapy. On the other hand, if a patient is not a failure (2^{nd} value) and is a success (1^{st} value) and the 3^{rd} value improves progressively throughout the days of evaluation, then the system implementing the algorithm described above can output for the physician a determination that the patient has responded to a given therapy. Another output of the algorithm is a prediction of the survival function outcome for the subject. The prediction can be of a 60 day outcome indicating the likelihood of re-hospitalization or death for that subject, or a group of subjects, by day-60 after the baseline measurement and treatment.

Empirical data supports the predictive nature of the methodology described herein. In particular, data captured from a sample of 305 subjects was provided to an algorithm programmed as described above. The results are described next.

### EXAMPLE

### Day 60 Outcomes by Success and Dyspnea Improvement in the Pilot Phase

Subjects were classified as “treatment success” if they reported moderately to markedly better dyspnea on Days 2 and 3 in the absence of “treatment failure.” Treatment failure here means death, worsening heart failure, heart failure readmission by Day 7, or persistent renal impairment defined as a 0.3 mg/dL or more increase in serum creatinine from baseline to Day 7 confirmed at Day 14.

Analysis of outcomes was conducted both by treatment success as defined for the three-category outcome, and by moderately-markedly better dyspnea on Days 2 and 3. The classification of subjects by these two definitions of dyspnea “success” is given in the table below:

| | Moderately-markedly better | | | |
|---|---|---|---|---|
| Ordered | dyspnea on Days 2 and 3 | | | |
| outcome | Missing | No | Yes | Total |
| Missing | 1 | 0 | 0 | |
| Treatment | 1 | 38 (27%) | 29 (18%) | 67 |
| failure | | | | |
| No change | 0 | 102 (73%) | 1 (1%) | 103 |
| Treatment | 0 | 0 (0%) | 133 (81%) | 133 |
| success | | | | |
| Total | | 140 (100%) | 163 (100%) | 303 |

304 of 305 subjects enrolled in the pilot phase were included in the analysis of outcomes by treatment success (one subject was missing data such that a classification could not be made). 303 subjects could be included in the analysis by dyspnea improvement. 30 (19%) of the 163 subjects who reported moderately to markedly better dyspnea on Days 2 and 3 did not otherwise meet the criteria for treatment success: 1 was classified as no change and the other as treatment failure.

27 of the 304 subjects died, and 96 died or were re-hospitalized by Day 60. Average follow-up was 56.7 days.

Results show that treatment success in the absence of treatment failure is a strong predictor of both death and death or re-hospitalization by Day 60, and is more strongly associated with Day 60 outcomes than dyspnea improvement alone.

Exclusion of 2 deaths on or before Day 7 (both with dyspnea improvement but subsequent failures), 6 subjects re-hospitalized on or before Day 7 (4 with dyspnea improvement but subsequent failures and 3 both without dyspnea improvement and treatment failure), and 2 subjects with follow-up on or before Day 7 (both treatment failures, 1 missing the dyspnea assessment) did not substantially change the results.

Predictor Results: Day 60 Outcomes by Treatment Success

| | | | Hazard ratio (95% |
|---|---|---|---|
| Kaplan-Meier estimates of | Treatment | | confidence interval |
| event | Success | | using Cox proportional |
| Rates | No | Yes | hazards model; “CI”) |
| Death by Day 60 | 12.6% | 4.6% | 0.34 (0.14-0.85) |
| Death or rehospitalization by | 40.7% | 21.2% | 0.46 (0.30-0.71) |
| Day 60 | | | |

COMPARISON: Day 60 Outcomes Only in View of Dyspnea Improvement on Days 2 and 3

Dyspnea improvement by itself (as the direct outcome of the questionnaire) has been determined to be a weak predictor of adverse outcomes, and hence less useful than “success” determination described above. This can be appreciated horn the table below.

| | Moderately-markedly | | |
|---|---|---|---|
| Kaplan-Meier estimates of | better dyspnea on | | |
| event | Days 2 and 3 | | Hazard ratio |
| Rates | No | Yes | (95% CI) |
| Death by Day 60 | 12.5% | 6.2% | 0.48 (0.22-1.06) |
| Death or rehospitalization by | 36.5% | 28.4% | 0.76 (0.51-1.13) |
| Day 60 | | | |

### Predictor Results: Day 60 Outcomes by Treatment Failure in the Pilot Phase

“Treatment failure” is defined as in the example above, namely, as death, worsening heart failure, heart failure readmission by Day 7, or persistent renal impairment defined as a 0.3 mg/dL or more increase in serum creatinine from baseline to Day 7 confirmed at Day 14.

68 (22%) of 304 enrolled subjects who could be classified were classified as having treatment failure. 27 of the 304 subjects died, and 96 died or were re-hospitalized by Day 60. Average follow-up was 56.7 days.

Accordingly, the results show that treatment failure is a strong predictor of both death and death or re-hospitalization by Day 60.

Exclusion of 2 deaths on or before Day 7 (both treatment failures), 6 subjects re-hospitalized on or before Day 7 (all treatment failures), and 2 subjects with follow-up on or before Day 7 (both treatment failures) did not substantially change the results.

| Kaplan-Meier estimates of | | | |
|---|---|---|---|
| event | Treatment Failure | | |
| Rates | No | Yes | Hazard ratio (95% CI) |
| Death by Day 60 | 6.8% | 16.9% | 2.66 (1.23-5.73) |
| Death or re-hospitalization by | 25.2% | 56.6% | 2.98 (1.97-4.50) |
| Day 60 | | | |

Referring now to FIG. 3 is a block diagram of a computer system 300 configured for employment of method 100. System 300 includes a user interface 305, a processor 310, and a memory 315. System 300 may be implemented on a general purpose microcomputer, such as one of the members of the Sun® Microsystems family of computer systems, one of the members of the IBM® Personal Computer family, one of the members of the Apple® Computer family, or a myriad other conventional workstation, desktop computer, laptop computer, a netbook computer, a personal digital assistant, or a smart phone. Although system 300 is represented herein as a standalone system, it is not limited to such, but instead can be coupled to other computer systems via a network (not shown).

Memory 315 is a memory for storing data and instructions suitable for controlling the operation of processor 310. An implementation of memory 315 would include a random access memory (RAM), a hard drive and a read only memory (ROM). One of the components stored in memory 315 is a program 320.

Program 320 includes instructions for controlling processor 310 to execute method 100. Program 320 may be implemented as a single module or as a plurality of modules that operate in cooperation with one another. Program 320 is contemplated as representing a software embodiment of the method described hereinabove.

User interface 305 includes an input device, such as a keyboard, touch screen, tablet, or speech recognition subsystem, for enabling a user to communicate information and command selections to processor 310. User interface 305 also includes an output device such as a display or a printer. In the case of a touch screen, the input and output functions are provided by the same structure. A cursor control such as a mouse, track-ball, or joy stick, allows the user to manipulate a cursor on the display for communicating additional information and command selections to processor 310.

While program 320 is indicated as already loaded into memory 315, it may be configured on a storage media 325 for subsequent loading into memory 315. Storage media 325 can be any conventional storage media such as a magnetic tape, an optical storage media, a compact disc, or a floppy disc. Alternatively, storage media 325 can be a random access memory, or other type of electronic storage, located on a remote storage system.

The methods described herein have been indicated in connection with flow diagrams that facilitate a description of the principal processes; however, certain blocks can be invoked in an arbitrary order, such as when the events drive the program flow such as in an object-oriented program. Accordingly, the flow diagram is to be understood as an example flow and that the blocks can be invoked in a different order than as illustrated.

It should be understood that various combination, alternatives and modifications of the present invention could be devised by those skilled in the art. The present invention is intended to embrace all such alternatives, modifications and variances that fall within the scope of the appended claims.

We claim:

1. A method for monitoring a health status of a human subject, comprising the steps of:
capturing medical data concerning the health of the subject at defined intervals using a questionnaire, wherein the questionnaire provides a standard script for data capture, wherein a first part of the questionnaire has a first portion of the captured data on a Likert scale and a second portion on a visual analog scale, and wherein a second part of the questionnaire has the captured data in the form of an assessment by a physician of health symptoms of the subject;
inputting the captured data into a computer;
providing the captured data to an algorithm configured to assess a risk of acute heart failure;
computing the risk of acute heart failure by executing the algorithm in the computer using the captured data input from a plurality of the defined intervals; and
outputting to the physician the health status as being either improved or worsening as a function of a value of the computed risk.

2. The method of claim 1, wherein the intervals are spaced by unequal amounts of time.

3. The method of claim 2, wherein the first interval is about six hours from a first data capture, a second interval is about 6 hrs from the first interval, a third interval is about twelve hours the second interval, a fourth interval is about twenty-four hours from the third interval, and wherein a plurality of successive intervals are twenty-four hours apart.

4. The method of claim 1, wherein the standard script causes a sequential capture of medical data.

5. The method of claim 1, wherein the capturing and inputting steps are performed concurrently through an electronic data form at or after each defined interval.

6. A method of predicting a survival function outcome of a human subject, comprising the steps of:
capturing medical data concerning the health of the subject at defined intervals using a questionnaire, wherein the questionnaire provides a standard script for data capture, wherein a first part of the questionnaire has a first portion of the captured data on a Likert scale and a second portion on a visual analog scale, and wherein a second part of the questionnaire has the captured data in the form of an assessment by a physician of health symptoms of the subject;
inputting the captured data into a computer;
providing the captured data to an algorithm configured to assess a risk of acute heart failure;
computing the risk of acute heart failure by executing the algorithm in the computer using the captured data input from a plurality of the defined intervals; and
predicting the survival function outcome for the subject using the output of the algorithm.

7. The method of claim 6, wherein the intervals are spaced by unequal amounts of time.

8. The method of claim 7, wherein the first interval is about six hours from a first data capture, a second interval is about 6 hrs from the first interval, a third interval is about twelve hours the second interval, a fourth interval is about twenty-four hours from the third interval, and wherein a plurality of successive intervals are twenty-four hours apart.

9. The method of claim 6, wherein the standard script causes a sequential capture of medical data.

10. The method of claim 6, wherein the capturing and inputting steps are performed concurrently through an electronic data form at or after each defined interval.
